(19) United States
(12) Patent Application Publication
(10) Pub. No.: US 2007/0299243 A1
(43) Pub. Date: Dec. 27, 2007

(54) POLY(ARYLENE ETHER) PROCESS AND COMPOSITION

(76) Inventors: Erik R. Delsman, Breda (NL); Gert-Jan Schoenmakers, Prinsenbeek (NL); Paul Tock, Bergen op Zoom (NL)

Correspondence Address: CANTOR COLBURN LLP - GE PLASTICS-NORYL, 55 GRIFFIN ROAD SOUTH, BLOOMFIELD, CT, 06002, US

(21) Appl. No.: 11/425,741

(22) Filed: Jun. 22, 2006

(51) Int. Cl. C08F 6/22; C07C 43/20

(52) U.S. Cl. 528/492; 568/660

(57) ABSTRACT

A method of purifying a polymerization reaction mixture containing a poly(arylene ether) is described. The method uses multiple separations of organic and aqueous phases to achieve rapid and efficient removal of polymerization catalyst metal from the poly(arylene ether). When combined with particular precipitation conditions, the method produces a poly(arylene ether) having an improved combination of particle size distribution and low catalyst metal concentration.

### BACKGROUND OF THE INVENTION

Poly(arylene ether)s and their blends with styrenic resins are highly valued for their properties, including high impact strength, stiffness, heat resistance, and electrical resistance. Poly(arylene ether)s are typically prepared by oxidative polymerization of monohydric phenols in solution, a reaction that is catalyzed by metal amine complexes. The catalyst metal is then separated from the poly(arylene ether) by chelation with an aqueous chelant solution. See, for example, U.S. Pat. No. 3,733,301 to Modan, U.S. Pat. No. 3,783,147 to Calicchia et al., and U.S. Pat. No. 3,838,102 to Bennett et al. The catalyst metal content of the precipitated poly(arylene ether) may be further reduced, albeit slightly, by water washing of the precipitate. Even though existing processes are effective to remove the vast majority of catalyst metal from isolated poly(arylene ether), there remains a need for improved processes to further reduce the catalyst metal concentration in isolated poly(arylene ether). Furthermore, it is beneficial, for example for flexibility of plant operation, to reduce the residence time in the various pieces of equipment used.

### BRIEF DESCRIPTION OF THE INVENTION

One embodiment is a method of purifying a poly(arylene ether), comprising: combining a chelant with a poly(arylene ether) reaction mixture comprising a poly(arylene ether), an aromatic hydrocarbon solvent, water, and a catalyst metal; separating the combined chelant and poly(arylene ether) reaction mixture in a separation apparatus with an average residence time of less than or equal to 60 seconds to yield a first less dense phase comprising poly(arylene ether) and solvent and a first more dense phase comprising water and chelant; combining the first less dense phase with an aqueous solution; and separating the combined first less dense phase and aqueous solution in a separation apparatus with an average residence time of less than or equal to 60 seconds to yield a second less dense phase comprising poly(arylene ether) and solvent and a second more dense phase comprising water.

One embodiment is a method of purifying a poly(arylene ether), comprising: combining a chelant with a poly(arylene ether) reaction mixture comprising toluene, copper catalyst metal, and a poly(arylene ether) comprising 2,6-dimethyl-1,4-phenylene ether units; wherein the chelant is provided as a chelant solution comprising an alkali metal salt of nitrilotriacetic acid; separating the combined chelant and poly(arylene ether) reaction mixture in a liquid-liquid centrifuge with an average residence time of about 4 to about 40 seconds to yield a first less dense phase comprising poly(arylene ether) and solvent and a first more dense phase comprising water and chelant; combining the first less dense phase with an aqueous solution consisting of water in about 2 to about 60 seconds; and separating the combined first less dense phase and aqueous solution in about 4 to about 40 seconds with a liquid-liquid centrifuge to yield a second less dense phase comprising poly(arylene ether) and solvent and a second more dense phase comprising water.

One embodiment is a method of purifying a poly(arylene ether), comprising: combining a chelant with a poly(arylene ether) reaction mixture comprising toluene, copper catalyst metal, and a poly(arylene ether) comprising 2,6-dimethyl-1,4-phenylene ether units; wherein the chelant is provided as a chelant solution comprising an alkali metal salt of nitrilotriacetic acid; separating the combined chelant and poly(arylene ether) reaction mixture in a liquid-liquid centrifuge with an average residence time of about 4 to about 15 seconds to yield a first less dense phase comprising poly(arylene ether) and solvent and a first more dense phase comprising water and chelant; wherein the liquid-liquid centrifuge is operated at a light phase back pressure of about 50 to about 300 kilopascals; combining the first less dense phase with an aqueous solution consisting of water in about 2 to about 60 seconds; wherein the aqueous solution is used in an amount of about 1 to about 8 weight percent, based on the weight of the first less dense phase; and separating the combined first less dense phase and aqueous solution in a liquid-liquid centrifuge with an average residence time of about 4 to about 15 seconds to yield a second less dense phase comprising poly(arylene ether) and solvent and a second more dense phase comprising water; wherein the liquid-liquid centrifuge is operated at a light phase back pressure of about 50 to about 300 kilopascals.

Other embodiments, including poly(arylene ether)s prepared by the above methods, are described in detail below.

### BRIEF DESCRIPTION OF THE DRAWINGS

Referring now to the drawings wherein like elements are numbered alike in several FIGURES:

FIG. 1 is a schematic depiction of an apparatus 10 for the preparation of a poly(arylene ether), wherein the apparatus 10 comprises a reactor 20, a separator 50, a mixer 60, a second separator 70, and an isolation module 80.

FIG. 2 is a schematic depiction of an apparatus for the preparation of a poly(arylene ether), wherein the apparatus 100 comprises a reactor 20, a holding tank 30, a separator 50, a mixer 60, a second separator 70, and an isolation module 80.

FIG. 3 is a schematic depiction of an apparatus for the preparation of a poly(arylene ether), wherein the apparatus 200 comprises a reactor 20, a separator feed tank 40, a separator 50, a mixer 60, a second separator 70, and an isolation module 80.

FIG. 4 is a schematic depiction of an apparatus for the preparation of a poly(arylene ether), wherein the apparatus 300 comprises a reactor 20, a holding tank 30, a separator feed tank 40, a separator 50, a mixer 60, a second separator 70, and an isolation module 80.

FIG. 5 is a schematic depiction of an apparatus for the preparation of a poly(arylene ether), wherein the apparatus 400 comprises a reactor 20, a holding tank 30, a separator feed tank 40, a separator 50, and an isolation module 80.

### DETAILED DESCRIPTION OF THE INVENTION

U.S. Application Publication No. US 2005/0171331 A1 of Ingelbrecht describes a particular method of precipitating a poly(arylene ether) from solution to produce a poly(arylene ether) powder. Compared to previously known processes, the Ingelbrecht process substantially reduces the content of very small particles (for example, those having particle sizes less than 75 micrometers), thereby reducing the difficulty and expense of handing the poly(arylene ether) powder. However, implementation of this improved precipitation process had the unexpected consequence of increasing the concentration of catalyst metal in the poly(arylene ether) powder. This increased catalyst metal content was undesirable, because increased catalyst metal content has been correlated with decreased long-term oxidative stability of the poly(arylene ether). The present inventors therefore conducted extensive research to discover a poly(arylene ether) production method that would produce a product poly(arylene ether) powder having both a low content of small particles and a reduced content of catalyst metal.

One embodiment is a method of purifying a poly(arylene ether), comprising: combining a chelant with a poly(arylene ether) reaction mixture comprising a poly(arylene ether), an aromatic hydrocarbon solvent, water, and a catalyst metal; separating the combined chelant and poly(arylene ether) reaction mixture in a separation apparatus with an average residence time of less than or equal to 60 seconds to yield a first less dense phase comprising poly(arylene ether) and solvent and a first more dense phase comprising water and chelant; combining the first less dense phase with an aqueous solution; and separating the combined first less dense phase and aqueous solution in a separation apparatus with an average residence time of less than or equal to 60 seconds to yield a second less dense phase comprising poly(arylene ether) and solvent and a second more dense phase comprising water.

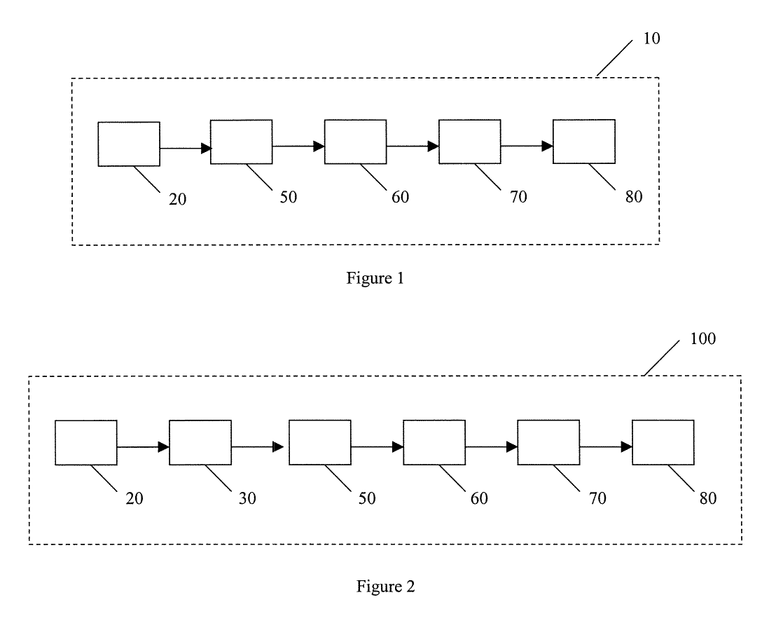

One apparatus suitable for carrying out the method is schematically depicted in FIG. 1, where a first apparatus 10 includes a reactor 20, a separator 50, a mixer 60, a second separator 70, and an isolation module 80 that may include apparatus for isolating the poly(arylene ether) as a solid. In general, the poly(arylene ether) is formed via an oxidative polymerization reaction in reactor 20. The product of this reaction is a poly(arylene ether) reaction mixture that comprises dissolved poly(arylene ether), a solvent, water (a by-product of the oxidative polymerization reaction), and a catalyst metal. Chelant is combined with the poly(arylene ether) reaction mixture, for example when that mixture is in the reactor 20, or during its transfer to separator 50. The combined chelant and poly(arylene ether) are transferred through the separator 50. The separator 50 effects separation of a first light phase containing the poly(arylene ether) and solvent, and a first heavy phase containing water and chelated catalyst metal. The first light phase is blended with an aqueous solution and transferred to mixer 60, where the two phases are mixed. The combined phases are then transferred to second separator 70, where they are separated into a second light phase containing the poly(arylene ether) and solvent and a second heavy phase containing water and additional chelated catalyst metal. The second light phase is then transferred to the isolation module 80, where the poly(arylene ether) is isolated in solid form. Suitable isolation methods include, for example, solution concentration, devolatilizing extrusion, spray drying, precipitation, filtration, washing, and drying.

Another apparatus suitable for carrying out the method is schematically depicted in FIG. 2, where a second apparatus 100 includes the same components as the first apparatus 10 in FIG. 1 and further includes a holding tank 30, which is an alternate position for combining and mixing the chelant and the poly(arylene ether) reaction mixture. When this apparatus is employed, the poly(arylene ether) reaction mixture (with or without combined chelant) is transferred to holding tank 30. The combined chelant and reaction mixture are transferred from holding tank 30 to separator 50.

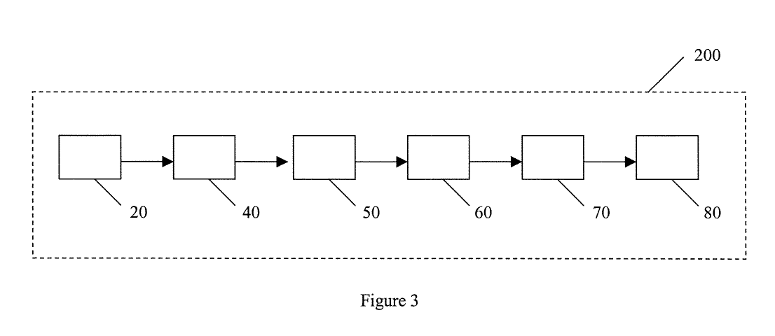

Another apparatus suitable for carrying out the method is schematically depicted in FIG. 3, where a third apparatus 200 includes the same components as the first apparatus 10 in FIG. 1 and further includes a separator feed tank 40, which facilitates transfer of the combined chelant and reaction mixture to the separator 50. If the separator 50 is a liquid-liquid centrifuge or other separation apparatus capable of operating continuously, transfer of the combined chelant and reaction mixture from the separator feed tank 40 to the separator 50 may be continuous.

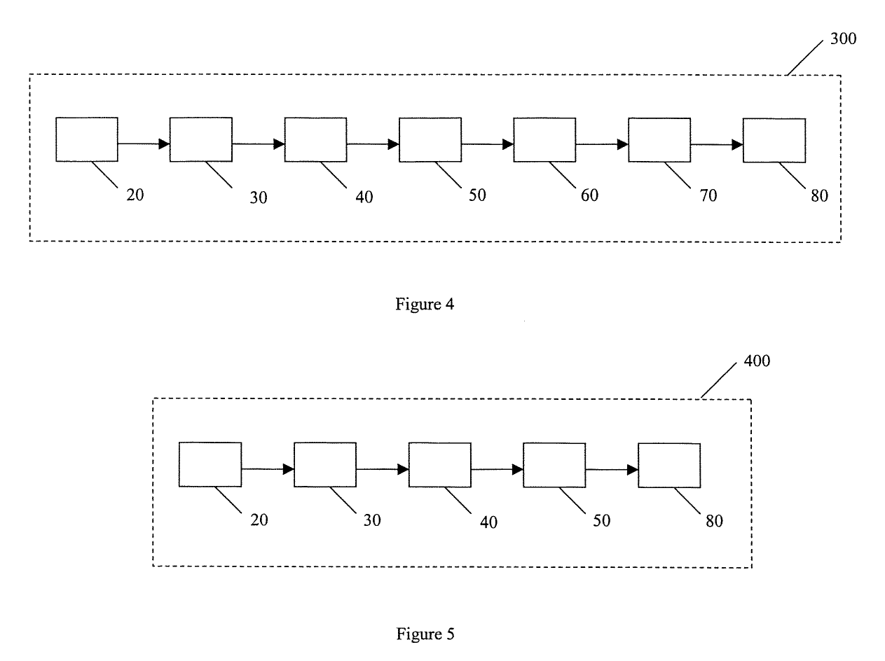

Another apparatus suitable for carrying out the method is schematically depicted in FIG. 4, where a fourth apparatus 300 includes the same components as the first apparatus 10 in FIG. 1 and further includes both a holding tank 30 and a separator feed tank 40. When this apparatus is used, chelant may be combined with the poly(arylene ether) reaction mixture when that mixture is in the reactor 20, or during its transfer to holding tank 30, or after it has been transferred to the holding tank 30. The combined chelant and poly(arylene ether) are mixed in reactor 20 and/or in holding tank 30, then transferred to separator feed tank 40, from which the combined mixture is continuously transferred through the separator 50. The separator 50 effects separation of a first light phase containing the poly(arylene ether) and solvent, and a first heavy phase containing water and chelated catalyst metal. The first light phase is blended with an aqueous solution and transferred to mixer 60, where the two phases are mixed. The combined phases are then transferred to second separator 70, where they are separated into a second light phase containing the poly(arylene ether) and solvent and a second heavy phase containing water and additional chelated catalyst metal. The second light phase is then transferred to the isolation module 80, where the poly(arylene ether) is isolated in solid form.

A fifth apparatus, depicted in FIG. 5, is described below in Examples 1-5.

The method is applicable to poly(arylene ether) reaction mixtures having a wide range of concentrations. For example, the method is applicable to poly(arylene ether) reaction mixtures that are the direct product of oxidative polymerization of at least one monohydric phenol, optionally in combination with at least one dihydric and/or polyhydric phenol. Such reaction mixtures typically have a poly(arylene ether) concentration of about 10 to about 40 weight percent, based on the total weight of the poly(arylene ether) reaction mixture.

The method includes combining a chelant with the poly(arylene ether) reaction mixture. The chelant may be provided in pure form (for example, as a pure solid). Alternatively, the chelant may be provided as a “chelant solution” that comprises the chelant and a suitable solvent for the chelant, such as, for example, water. The latter approach has the advantage of avoiding delays associated with dissolution of solid chelant. When used, the “chelant solution” may have a wide range of chelant concentrations. For example, the chelant solution may comprise about 5 to about 50 weight percent chelant, based on the total weight of the chelant solution. Within this range, the chelant solution may comprise at least about 10 weight percent chelant. It is also possible to use a wide range of chelant solution amounts, relative to the poly(arylene ether) reaction mixture. For example, in some embodiments, the chelant solution may be used in an amount of about 0.05 to about 5 weight percent, based on the weight of the poly(arylene ether) reaction mixture. Within this range, the amount may be at least about 0.1 weight percent. Also within this range, the amount may be up to about 3 weight percent, or up to about 2 weight percent, or up to about 1 weight percent, or up to about 0.5 weight percent. The molar amount of chelant is generally at least about one mole per mole of catalyst metal to be chelated. For example, in some embodiments, a chelant amount of about 0.95 to about 4 moles chelant per mole of catalyst metal may be used. However, efficient chelation may be achieved with little or no molar excess of chelant, that is, by using close to 1 mole of chelant per mole of catalyst metal added to the polymerization reaction mixture.

The present inventors have found that chelation of the catalyst metal depends significantly on the efficiency of mixing the chelant and the poly(arylene ether) reaction mixture. In one embodiment, the poly(arylene ether) reaction mixture and the chelant solution are combined in a stirred tank having a stirrer Reynolds number of about 10,000 to about 60,000. In general, a Reynolds number for a stirred tank may be calculated based on the tank diameter or the stirrer tip-to-tip diameter. In this embodiment, the Reynolds number is calculated based on the stirrer tip-to-tip diameter. The extent of mixing may also be expressed as a mixing power or mixing energy. Thus, in some embodiments, the chelant and the poly(arylene ether) reaction mixture are combined with a mixing power input of about 0.1 to about 10 Watt per kilogram total of poly(arylene ether) reaction mixture and chelant (including any solvent used to dilute the chelant). In some embodiments, the chelant and the poly(arylene ether) reaction mixture are combined with a mixing energy of about 1 to about 10 kilojoules per kilogram total of poly(arylene ether) reaction mixture and chelant (including any solvent used to dilute the chelant). One skilled in the art knows how to calculate mixing energy and mixing power.

The present inventors have also found that the catalyst metal concentration in the final poly(arylene ether) may be reduced by combining the chelant and the poly(arylene ether) reaction mixture in the polymerization reactor 20, rather than the holding tank 30 or the separator feed tank 40. Surprising, it has been found that addition of the chelant to the polymerization reactor 20 has no adverse effect on subsequent polymerizations conducted in reactor 20. However, under some circumstances, the benefit of early addition of chelant to the poly(arylene ether) reaction mixture may be small, and it may be preferred to combine the chelant and the poly(arylene ether) reaction mixture in the holding tank 30, rather than the polymerization reactor 20. Alternatively, the chelant and poly(arylene ether) reaction mixture may be combined between the polymerization reactor 20 and the holding tank 30.

In some embodiments, water is combined with the poly(arylene ether) reaction mixture separately from combining the reaction mixture with chelant. This combination with water may occur before, during, or after combination with chelant.

When the chelant is provided in the form of a chelant solution, it may be desirable to adjust the temperature of the poly(arylene ether) reaction mixture and/or the chelant solution prior to combining them. For example, in some embodiments, the temperature of the poly(arylene ether) reaction mixture is about 40 to about 60° C. when it is combined (that is, immediately before it is combined) with the chelant solution. It may also be desirable to adjust the temperature of the combined poly(arylene ether) reaction mixture and the chelant solution during the first separation step. For example, in some embodiments, the temperature of the combined poly(arylene ether) reaction mixture and chelant solution is about 50 to about 70° C. during the first separation step.

The method comprises separating the combined chelant and poly(arylene ether) reaction mixture in a separation apparatus with an average residence time of less than or equal to 60 seconds to yield a first less dense phase comprising poly(arylene ether) and solvent and a first more dense phase comprising water and chelant. The term “separating” means that a first less dense phase and a first more dense phase are produced. It does not require complete separation of chelant from poly(arylene ether). In some embodiments, the separation is effective to produce a first less dense phase comprising less than 15 weight percent water, or less than 10 weight percent water, or less than 5 weight percent water, based on the weight of the first less dense phase.

In some embodiments, the average residence time is significantly lower than 60 seconds. For example, the average residence time may be less than about 40 seconds, or less than about 30 seconds, or less than about 20 seconds, or less than about 10 seconds. In some embodiments, the average residence time may be as low as about 4 seconds are effective. One skilled in the art knows how to calculate the average residence time of a separator based on the steady state process flow rate and the separator work capacity.

After the combined chelant and poly(arylene ether) reaction mixture solution is separated to yield a first less dense phase and a first more dense phase, the first less dense phase is combined with an aqueous solution. Combining the first less dense phase and the aqueous solution may occur, for example, in mixer 60 of FIG. 1. There is no particular limit on amount of aqueous solution used. In some embodiments, the aqueous solution is used in an amount of about 1 to about 8 weight percent, based on the weight of the first less dense phase. Within this range, the amount of aqueous solution may be at least about 2 weight percent, at least about 3 weight percent. Also within this range, the amount of aqueous solution may be up to about 6 weight percent.

In some embodiments, the aqueous solution is substantially free of chelant. In this context, “substantially free” means that the solution comprises less than 0.1 weight percent of chelant. In some embodiments, the aqueous solution is free of any intentionally added chelant. In some embodiments, the aqueous solution consists of water.

It may be desirable to adjust the temperature of the first less dense phase and/or the aqueous solution prior to combining them in mixer 60. For example, in some embodiments, the aqueous solution has a temperature of about 50 to about 70° C. when it is combined (that is, immediately before it is combined) with the first less dense phase. In some embodiments the temperature of the aqueous solution may be within about 20° C., or within about 10° C., of the temperature of the first less dense phase when the two are combined in mixer 60 (that is, immediately before the two phases are combined).

It has been found that particular conditions for mixing the aqueous solution and the first less dense phase may favor catalyst metal removal. For example, efficient catalyst metal removal has been observed when combining a first less dense phase flow having a Reynolds number of about 1,000 to about 4,000 with an aqueous solution flow having a Reynolds number of about 100 to about 400, especially when the ratio of the Reynolds number of the first less dense phase flow to the Reynolds number of the aqueous solution flow is about 5 to about 20.

The first less dense phase and the aqueous solution may be combined rapidly in mixer 60. For example, in some embodiments, combining the first less dense phase with the aqueous solution is conducted in about 2 to about 60 seconds. Within this range, the time may be up to about 30 seconds, or up to about 10 seconds, or up to about 7 seconds.

There is no particular limit on the method used to combine the first less dense phase and the aqueous solution. In one embodiment, mixer 60 used to combine the first less dense phase and the aqueous solution may be a static mixer. In another embodiment, mixer 60 may be a dynamic mixer. In another embodiment, mixer 60 may be a stirred tank, with or without external circulation. In another embodiment, the first less dense phase and the aqueous solution may be combined without intentional mixing, relying instead on mixing that occurs within the subsequent separation step.

The combined first less dense phase and aqueous solution may be rapidly and efficiently separated. For example, in some embodiments, separating the combined first less dense phase and aqueous solution is conducted in about 4 to about 60 seconds. In some embodiments, the separation time is significantly lower. For example, the separation time may be less than or equal to about 40 seconds, or less than or equal to about 30 seconds, or less than or equal to about 20 seconds, or less than or equal to about 10 seconds. In some embodiments, separation times at least as low as about 4 seconds are effective.

The separation steps (separation of the combined chelant and poly(arylene ether) reaction mixture, and separation of the combined first less dense phase and aqueous solution) may be effected using liquid-liquid separation apparatus known in the art. In some embodiments, separating the combined chelant and poly(arylene ether) reaction mixture and/or separating the combined first less dense phase and aqueous solution comprises using a liquid-liquid centrifuge. Suitable liquid-liquid centrifuges are described, for example, in U.S. Pat. No. 2,622,797 of Hemfort, U.S. Pat. No. 4,614,598 of Zettier et al., and U.S. Pat. No. 4,755,165 of Gunnewig, and in Great Britain Patent Specification No. 884,768. Suitable liquid-liquid centrifuges are also commercially available, for example from GEA-Westfalia Separator AG. Liquid-liquid centrifuges are particularly useful for continuous separation processes. Other suitable separation apparatus includes coalescers, decanters, and the like. Suitable coalescers are described, for example, in U.S. Pat. No. 6,332,987 B1 to Whitney et al., and U.S. Patent Application Publication No. US 2005/0178718 A1 of Geibel et al.

The present inventors have unexpectedly discovered that operating a liquid-liquid centrifuge at increased light phase back pressure significantly improves the separation of light and heavy phases. Thus, in one embodiment, at least one of separating the combined chelant and poly(arylene ether) reaction mixture and separating the combined first less dense phase and aqueous solution comprises operating a liquid-liquid centrifuge at a light phase back pressure of about 50 to about 300 kilopascals. Within this range, the light phase back pressure may be at least about 100 kilopascals. Also within this range, the light phase back pressure may be up to about 200 kilopascals.

The method provides efficient removal of catalyst metal from poly(arylene ether) while producing very little aqueous waste. Typically, the aqueous waste must be treated to remove catalyst metal (for example, by precipitation). The amount of aqueous waste generated may be further reduced by recycling at least a portion of the second more dense phase generated in the second separation step. For example, when the chelant is provided as a chelant solution, some embodiments comprise recycling at least 30, at least 40, or at least 50 weight percent of the second more dense phase for use as at least a portion of the chelant solution. Alternatively, when the chelant is provided as a solid and water is separately combined with the poly(arylene ether) reaction mixture (separately from the combination with chelant), some embodiments comprise recycling at least 30, at least 40, or at least 50 weight percent of the second more dense phase for use as at least a portion of the water combined with the poly(arylene ether) reaction mixture.

The method is beneficially practiced on poly(arylene ether) reaction mixtures that contain little or no solid poly(arylene ether). In some embodiments, at least 90, at least 95, or at least 98 weight percent of the poly(arylene ether) is dissolved in the aromatic hydrocarbon solvent when the chelant is combined with the poly(arylene ether) reaction mixture. In other words, in some embodiments, substantially all of the poly(arylene ether) is in solution when the reaction mixture and chelant are combined. This contrasts with so-called reactive precipitation processes, in which a substantial fraction of the poly(arylene ether) formed by oxidative polymerization precipitates from the reaction solution.

In one embodiment, the chelant solution and the aqueous solution are substantially free of reducing agents such as sulfite, dithionite, and hydrazine. In another embodiment, the chelant solution and/or the aqueous solution comprises a reducing agent such as sulfite, dithionite, hydrazine, or a combination thereof.

The method is extremely effective at reducing the catalyst metal concentration in the second less dense phase. Thus, in some embodiments, the second less dense phase comprises the catalyst metal in a concentration of about 0.1 to about 2 parts per million by weight, based on the total weight of the second less dense phase. Within this range, the catalyst metal concentration may be up to about 1 part per million by weight (ppm), or up to about 0.5 ppm. The catalyst metal concentration of the second less dense phase may also be expressed relative to the catalyst metal concentration of the poly(arylene ether) reaction mixture. Thus, in some embodiments, the ratio of the catalyst metal concentration in the second less dense phase to the catalyst metal concentration in the poly(arylene ether) reaction mixture is about 1:500 to about 1:50.

The second less dense phase produced by the method is suitable for use as a feedstock for precipitation methods that produce low levels of very small particles. Thus, in some embodiments, the method further comprises isolating the poly(arylene ether) in a powder form comprising less than 10 weight percent of particles smaller than 38 micrometers and less than 2 parts per million by weight of the catalyst metal. Poly(arylene ether) precipitation methods capable of producing low levels of very small particles (but not capable of producing the low catalyst metal concentrations of the present process) are described, for example, in U.S. Patent Application Publication No. 2005/0171331 A1 of Ingelbrecht. Thus, one embodiment is a poly(arylene ether) powder having these particle size characteristics and further having the low catalyst metal concentration enabled by the present methods.

The method is applicable to poly(arylene ether)s having a wide variety of structures. In some embodiments, the poly(arylene ether) comprises repeating structural units having the formula

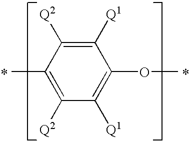

wherein for each structural unit, each Q¹ is independently halogen, unsubstituted or substituted C₁-C₁₂ hydrocarbyl with the proviso that the hydrocarbyl group is not tertiary hydrocarbyl, C₁-C₁₂ hydrocarbylthio, C₁-C₁₂ hydrocarbyloxy, or C₂-C₁₂ halohydrocarbyloxy wherein at least two carbon atoms separate the halogen and oxygen atoms; and each Q² is independently hydrogen, halogen, unsubstituted or substituted C₁-C₁₂ hydrocarbyl with the proviso that the hydrocarbyl group is not tertiary hydrocarbyl, C₁-C₁₂ hydrocarbylthio, C₁-C₁₂ hydrocarbyloxy, or C₂-C₁₂ halohydrocarbyloxy wherein at least two carbon atoms separate the halogen and oxygen atoms. The asterisks in the structure above represent points of attachment to the remainder of the poly(arylene ether) molecule. As used herein, the term “hydrocarbyl”, whether used by itself, or as a prefix, suffix, or fragment of another term, refers to a residue that contains only carbon and hydrogen. The residue may be aliphatic or aromatic, straight-chain, cyclic, bicyclic, branched, saturated, or unsaturated. It may also contain combinations of aliphatic, aromatic, straight chain, cyclic, bicyclic, branched, saturated, and unsaturated hydrocarbon moieties. The hydrocarbyl residue, when so stated however, may contain heteroatoms over and above the carbon and hydrogen members of the substituent residue. Thus, when specifically noted as containing such heteroatoms, the hydrocarbyl or hydrocarbylene residue may also contain carbonyl groups, amino groups, hydroxyl groups, or the like, or it may contain heteroatoms within the backbone of the hydrocarbyl residue. In some embodiments, the poly(arylene ether) comprises 2,6-dimethyl-1,4-phenylene ether units, 2,3,6-trimethyl-1,4-phenylene ether units, or a combination thereof.

The poly(arylene ether) may be a so-called bifunctional poly(arylene ether). Such poly(arylene ether)s comprise, on average, close to two terminal hydroxyl groups per molecule. For example, in one embodiment, the poly(arylene ether) comprises a bifunctional poly(arylene ether) having the structure

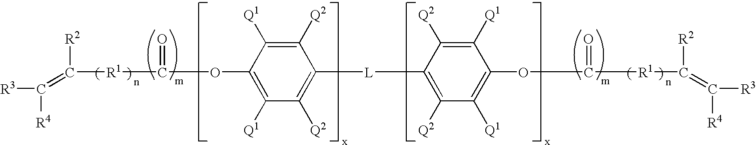

wherein each occurrence of Q¹ is independently halogen, unsubstituted or substituted C₁-C₁₂ hydrocarbyl with the proviso that the hydrocarbyl group is not tertiary hydrocarbyl, C₁-C₁₂ hydrocarbylthio, C₁-C₁₂ hydrocarbyloxy, or C₂-C₁₂ halohydrocarbyloxy wherein at least two carbon atoms separate the halogen and oxygen atoms; each occurrence of Q² is independently hydrogen, halogen, unsubstituted or substituted C₁-C₁₂ hydrocarbyl with the proviso that the hydrocarbyl group is not tertiary hydrocarbyl, C₁-C₁₂ hydrocarbylthio, C₁-C₁₂ hydrocarbyloxy, or C₂-C₁₂ halohydrocarbyloxy wherein at least two carbon atoms separate the halogen and oxygen atoms; each occurrence of x is independently 0 to about 100, with the proviso that the sum of each occurrence of x is at least three; each occurrence of R¹ is C₁-C₁₂ hydrocarbylene; each occurrence of m is independently 0 or 1; each occurrence of n is independently 0 or 1; each occurrence of R²-R⁴ is independently hydrogen or C₁-C₁₈ hydrocarbyl; and L has the structure

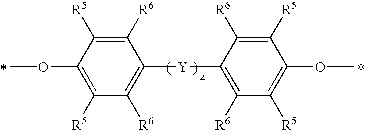

wherein each occurrence of R⁵ and R⁶ is independently hydrogen, halogen, unsubstituted or substituted C₁-C₁₂ hydrocarbyl with the proviso that the hydrocarbyl group is not tertiary hydrocarbyl, C₁-C₁₂ hydrocarbylthio, C₁-C₁₂ hydrocarbyloxy, or C₂-C₁₂ halohydrocarbyloxy wherein at least two carbon atoms separate the halogen and oxygen atoms; z is 0 or 1; and Y has the structure

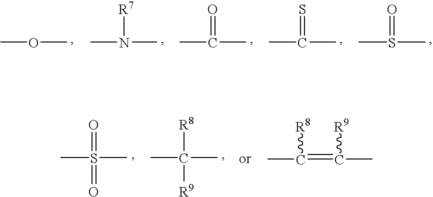

wherein R⁷, R⁸, and R⁹ are each independently hydrogen, C₁-C₁₂ hydrocarbyl, or the like. In the last substructure above, R⁸ and R⁹ may be disposed either cis or trans about the double bond. In one embodiment, the sum of each occurrence of x is at least 4. In some embodiments, the poly(arylene ether) is the product of a process comprising oxidative copolymerization of monomers comprising 2,6-dimethylphenol and 2,2-bis(4-hydroxy-2,6-dimethylphenyl)propane (“tetramethyl bisphenol A”).

The aromatic hydrocarbon solvent present in the poly(arylene ether) reaction mixture may be chosen from, for example, benzene, toluene, xylenes, chlorobenzene, dichlorobenzenes, trichlorobenzenes, tetrachlorobenzenes, pentachlorobenzene, hexachlorobenzene, and combinations thereof. In one embodiment, the aromatic hydrocarbon solvent is toluene.

The catalyst metal may be any metal that is effective to catalyze the oxidative polymerization of phenols. Such metals include, for example, copper, manganese, cobalt, and mixtures thereof. In one embodiment, the catalyst metal is copper.

There is no particular limit on the type of chelant used, as long is it is effective to sequester the catalyst metal. In one embodiment, the chelant is selected from polyalkylenepolyamine polycarboxylic acids, aminopolycarboxylic acids, aminocarboxylic acids, polycarboxylic acids, alkali metal salts of the foregoing acids, alkaline earth metal salts of the foregoing acids, mixed alkali metal-alkaline earth metal salts of the foregoing acids, and combinations thereof. Specific suitable chelants include, for example, hydroxyethylethylenediaminetriacetic acid, diethylenetriaminepentaacetic acid, nitrilotriacetic acid, ethylenediaminetetraacetic acid, alkali metal salts of the foregoing acids, alkaline earth metal salts of the foregoing acids, and combinations thereof. In some embodiments, the chelant comprises an alkali metal salt of nitrilotriacetic acid.

One embodiment is a method of purifying a poly(arylene ether), comprising: combining a chelant with a poly(arylene ether) reaction mixture comprising toluene, copper catalyst metal, and a poly(arylene ether) comprising 2,6-dimethyl-1,4-phenylene ether units; wherein the chelant is provided as a chelant solution comprising an alkali metal salt of nitrilotriacetic acid; separating the combined chelant and poly(arylene ether) reaction mixture in a liquid-liquid centrifuge with an average residence time of about 4 to about 40 seconds to yield a first less dense phase comprising poly(arylene ether) and solvent and a first more dense phase comprising water and chelant; combining the first less dense phase with an aqueous solution consisting of water in about 2 to about 60 seconds; and separating the combined first less dense phase and aqueous solution in about 4 to about 40 seconds with a liquid-liquid centrifuge to yield a second less dense phase comprising poly(arylene ether) and solvent and a second more dense phase comprising water. In some embodiments, this method further comprises isolating the poly(arylene ether) from solution (for example, from the second less dense phase), wherein the poly(arylene ether) comprises less than 10 weight percent of particles smaller than 38 micrometers and less than or equal to 2 parts per million by weight of copper. The invention extends to the poly(arylene ether) prepared by this method.

One embodiment is a method of purifying a poly(arylene ether), comprising: combining a chelant with a poly(arylene ether) reaction mixture comprising toluene, copper catalyst metal, and a poly(arylene ether) comprising 2,6-dimethyl-1,4-phenylene ether units; wherein the chelant is provided as a chelant solution comprising an alkali metal salt of nitrilotriacetic acid; separating the combined chelant and poly(arylene ether) reaction mixture in a liquid-liquid centrifuge with an average residence time of about 4 to about 15 seconds to yield a first less dense phase comprising poly(arylene ether) and solvent and a first more dense phase comprising water and chelant; wherein the liquid-liquid centrifuge is operated at a light phase back pressure of about 50 to about 300 kilopascals; combining the first less dense phase with an aqueous solution consisting of water in about 2 to about 60 seconds; wherein the aqueous solution is used in an amount of about 1 to about 8 weight percent, based on the weight of the first less dense phase; and separating the combined first less dense phase and aqueous solution in a liquid-liquid centrifuge with an average residence time of about 4 to about 15 seconds to yield a second less dense phase comprising poly(arylene ether) and solvent and a second more dense phase comprising water; wherein the liquid-liquid centrifuge is operated at a light phase back pressure of about 50 to about 300 kilopascals. In some embodiments, this method further comprises isolating the poly(arylene ether) from solution (for example, from the second less dense phase), wherein the poly(arylene ether) comprises less than 10 weight percent of particles smaller than 38 micrometers and less than or equal to 2 parts per million by weight of copper. The invention extends to the poly(arylene ether) prepared by this method.

The invention is further illustrated by the following non-limiting examples.

### EXAMPLES 1-5

These examples illustrate the effects of four process factors on the catalyst metal concentration in the isolated poly(arylene ether) resin: water addition to the holding tank, addition of a chelant solution to the reactor rather than the holding tank, improved mixing in the holding tank, and increased light phase back pressure in the liquid-liquid centrifuges. A schematic representation of the general apparatus used is given in FIG. 5. The fifth apparatus 400 includes a reactor 20, a holding tank 30, a separator feed tank 40, a separator 50, and an isolation module 80. In general, the poly(arylene ether) is formed via an oxidative polymerization reaction in reactor 20. The product of this reaction is a poly(arylene ether) reaction mixture that comprises dissolved poly(arylene ether), a solvent, water produced as a by-product of the polymerization reaction, and a catalyst metal. A chelant may be combined with the poly(arylene ether) reaction mixture, for example when that mixture is in the reactor 20, or while it is being transferred to holding tank 30, or after it has been transferred to holding tank 30. The combined chelant and poly(arylene ether) reaction mixture are mixed in the holding tank 30, then transferred to the separator feed tank 40, from which the combined mixture is continuously transferred to the separator 50. The separator 50 effects separation of a first light phase containing the poly(arylene ether) and solvent, and a first heavy phase containing water and chelated catalyst metal. The first light phase is then transferred to the isolation module 80, where the poly(arylene ether) is isolated in solid form.

In the specific process employed here, the poly(arylene ether) was poly(2,6-dimethyl-1,4-phenylene ether), the solvent was toluene, the concentration of the poly(arylene ether) in the polymerization reaction mixture was 25 weight percent based on the total weight of the reaction mixture, the catalyst metal was copper, and the concentration of copper in the polymerization reaction mixtures was 130 parts per million by weight based on the total weight of the polymerization reaction mixture. The chelant was the sodium salt of nitrilotriacetic acid. The aqueous chelant solution consisted of water and 40 weight percent chelant, based on the total weight of the chelant solution. The chelant solution was added in an amount of 0.2 weight percent based on the weight of the poly(arylene ether) reaction mixture. The centrifuge was a liquid-liquid centrifuge commercially available from GEA Westfalia Separator AG. The residence time in the centrifuge was about 10 to 40 seconds. The poly(arylene ether) isolation process consisted of solution concentration, high-shear precipitation, washing, filtration, and drying, as described in Example 1 of U.S. Patent Application Publication No. US 2005/0171331 A1 of Ingelbrecht.

The first process variable (“Chelant added to reactor?” in Table 1), relates to the point of addition of the chelant solution. The chelant solution was added either to the reactor (“yes”) or to the holding tank (“no”). The second process variable (“Water added to holding tank?” in Table 1) relates to whether water (in addition to the water present in the chelant solution) was added to the holding tank (“yes”) or to the separator feed tank (“no”). When water was added, it was added in an amount of 0.9-1.2 weight percent based on the weight of the poly(arylene ether) reaction mixture. The third process variable relates to the type of mixing used in holding tank 30. The first light phase and the water were either combined using a high efficiency mixer (“yes”) or a turbine stirrer (“no”). The fourth process variable (“Increased light phase back pressure in both centrifuges?” in Table 1) relates to the light phase back pressure at which the first and second centrifuges were operated. This back pressure was either 200 kilopascals (“yes”) or 50 kilopascals (“no”). For each process variation, the resulting isolated poly(arylene ether) was analyzed by atomic absorption spectroscopy to determine its copper content. That atomic absorption analysis used a Perkin-Elmer Model 100 Atomic Absorption Spectrophotometer, standard samples containing 10, 20, and 50 ppm Cu (as cupric chloride) in chlorobenzene, and experimental samples containing 0.6 grams of isolated poly(arylene ether) dissolved in 20 milliliters of chlorobenzene. Each copper content value is expressed as a mean value plus or minus a standard deviation, reflecting analysis on approximately forty poly(arylene ether) samples per process run. Statistical analysis of the copper content results, which are presented in Table 1, shows that the relative influence of the four process factors on reducing copper content was, in decreasing order of importance, increased light phase back pressure, water addition to the holding tank, improved mixing in the holding tank, and addition of chelant to the reactor rather than the holding tank.

TABLE 1

| | Ex. 1 | Ex. 2 | Ex. 3 | Ex. 4 | Ex. 5 |
|---|---|---|---|---|---|
| Chelant added to | no | no | yes | no | no |
| reactor? | | | | | |
| Water added to | no | yes | yes | yes | no |
| holding tank? | | | | | |
| Increased mixing in | no | no | yes | yes | yes |
| holding tank? | | | | | |
| Increased light phase back | no | yes | yes | yes | yes |
| pressure in both centrifuges? | | | | | |
| Cu in PPE (ppm) | 11.9 ± 2.3 | 7.0 ± 1.4 | 7.0 ± 0.6 | 6.1 ± 0.6 | 7.6 ± 0.7 |

### EXAMPLES 6-9

These examples demonstrate that addition of chelant solution to the reactor rather than the holding tank has a stronger influence on reducing copper concentration in the isolated resin when the contact time between chelant solution and poly(arylene ether) reaction mixture in the reactor is increased. The poly(arylene ether) reaction mixture and the chelant solution were the same as those described for Examples 1-5, except that addition of the chelant solution, mixing, and centrifuging were conducted on a laboratory scale. The chelant solution was added either at the end of the polymerization reaction (“EOR” in Table 2), or thirty minutes after the end of the polymerization reaction (“EOR+30” in Table 2). After the combined poly(arylene ether) reaction mixture and chelant solution were separated via centrifuge, the copper content of the light phase was determined. Examples 6 and 8 are replicates, as are examples 7 and 9. The results, presented in Table 2, show that earlier addition of the chelant solution decreases the copper concentration in the light phase produced in the first centrifuge step. Other experiments not described here showed that addition of chelant solution to the reactor did not adversely affect subsequent polymerization reactions in the same reactor.

TABLE 2

| | Ex. 6 | Ex. 7 | Ex. 8 | Ex. 9 |
|---|---|---|---|---|
| NTA addition time | EOR | EOR + 30 | EOR | EOR + 30 |
| Cu in light phase (ppm) | 3.1 | 10.5 | 3.1 | 7.6 |

### EXAMPLES 10-23

These examples illustrate the effects of adding a second centrifugation step, increasing the light phase back pressure of the centrifugation step, increasing the dilution of residual water in the light phase by varying the amount of water added between two centrifuge steps, and decreasing the processing rate. The process apparatus with one centrifuge step and associated process is schematically diagrammed in FIG. 5 and described above in Examples 1-5. The process apparatus with two centrifuge steps is schematically diagrammed in FIG. 4. In FIG. 4, the poly(arylene ether) preparation apparatus 10 includes a reactor 20, an holding tank 30, a separator feed tank 40, a separator 50, a mixer 60, a second separator 70, and an isolation module 80 that may include apparatus for concentration of the poly(arylene ether) solution, and precipitation, washing, filtration, and drying of the poly(arylene ether) resin. In general, the poly(arylene ether) is formed via an oxidative polymerization reaction in reactor 20. The product of this reaction is a poly(arylene ether) reaction mixture that comprises dissolved poly(arylene ether), a solvent, and a catalyst metal. Chelant is added to the poly(arylene ether) reaction mixture, either when that reaction mixture is in the reactor 20 or after it has been transferred to the holding tank 30. The combined chelant and poly(arylene ether) reaction mixture are mixed in the holding tank 30, then transferred to the separator feed tank 40, from which the combined mixture is continuously transferred to the separator 50. The separator 50 effects separation of a first light phase containing the poly(arylene ether) and solvent, and a first heavy phase containing water and chelated catalyst metal. The first light phase is blended with water and transferred to mixer 60 (which in these experiments consisted of a parallel array of three six-element static mixers), where the two phases are mixed. The combined phases are then transferred to second separator 70 (which in these experiments consisted of a liquid-liquid centrifuge obtained from GEA Westfalia Separator AG), where they are separated into a second light phase containing the poly(arylene ether) and solvent and a second heavy phase containing water and additional chelated catalyst metal. The second light phase is then transferred to the isolation module 80, where the poly(arylene ether) is isolated in solid form via solution concentration, precipitation, filtration, washing, and drying.

The process variations are summarized in Table 3. Processes either used the two-centrifuge apparatus of FIG. 4 (“Two centrifuge steps?”=“yes”) or the one-centrifuge apparatus of FIG. 5 (“Two centrifuge steps?”=“no”). “Light phase back pressure (kPa)” refers to the light phase back pressure, in kilopascals, maintained at each centrifuge in the process. “Dilution factor” refers to the measured ratio of the volume of water added between centrifuge steps in the two-centrifuge process to the volume of water present in the first light phase produced by the separator 50. “Scaled process flow rate (L/h)” refers to the continuous flow rate for the process forward of the separator feed tank, scaled to the lowest process flow rate studied. “Cu in light phase (ppm)” refers to the copper concentration of the light phase produced by the last centrifuge in the process, expressed in parts per million by weight relative to the total weight of the light phase. For the process with one centrifuge, the copper concentration of the fluid fed to the centrifuge was 44 parts per million by weight. For the process with two centrifuges, the copper concentration of the light phase generated by the first centrifuge was 2.6 parts per million.

The results are presented in Table 3. For the process with one centrifuge, increased light phase back pressure is associated with decreased copper concentration in the light phase. The copper concentration is only mildly sensitive to the process flow rate. For the process with two centrifuges, increasing the dilution factor from 5 to 10 is associated with a substantial decrease in light phase copper concentration when the process is run at a scaled process flow rate of 4.67. However, the effect of increased dilution factor is much more modest at a scaled process flow rate of 2.0. So, the effect of dilution factor is interdependent with the effect of process flow rate; increasing the dilution factor is more beneficial at high process flow rates.

TABLE 3

| | Ex. 10 | Ex. 11 | Ex. 12 | Ex. 13 |
|---|---|---|---|---|
| Two centrifuge steps? | no | no | no | no |
| Light phase back pressure | 100 | 100 | 100 | 150 |
| (kPa) | | | | |
| Dilution Factor | NA | NA | NA | NA |
| Scaled process flow rate | 1.0 | 1.67 | 3.33 | 1.0 |
| Cu in light phase (ppm) | 2.3 | 2.2 | 2.1 | 1.5 |
| | Ex. 14 | Ex. 15 | Ex. 16 | Ex. 17 |
| Two centrifuge steps? | no | no | no | no |
| Light phase back pressure | 150 | 200 | 200 | 250 |
| (kPa) | | | | |
| Dilution Factor | NA | NA | NA | NA |
| Scaled process flow rate | 3.33 | 1.67 | 3.33 | 1.67 |
| Cu in light phase (ppm) | 1.8 | 1.4 | 2.0 | 1.3 |
| | Ex. 18 | Ex. 19 | Ex. 20 | Ex. 21 |
| Two centrifuge steps? | yes | yes | yes | yes |
| Light phase back pressure | 240 | 240 | 240 | 240 |
| (kPa) | | | | |
| Dilution Factor | 6 | 5 | 11 | 9 |
| Scaled process flow rate | 2.0 | 4.67 | 2.0 | 4.0 |
| Cu in light phase (ppm) | 0.7 | 1.6 | 0.6 | 0.7 |

| | Ex. 22 | Ex. 23 |
|---|---|---|
| Two centrifuge steps? | yes | yes |
| Light phase back pressure | 240 | 240 |
| (kPa) | | |
| Dilution Factor | 19 | 22 |
| Scaled process flow rate | 4.67 | 2.0 |
| Cu in light phase (ppm) | 0.6 | 0.5 |

NA = not applicable

### EXAMPLES 24-29

These examples illustrate the use of a second centrifuge as a clarifier. In other words, they illustrate a two-centrifuge process in which there is no aqueous solution added to the poly(arylene ether)-containing phase between the first and second centrifuges.

The process is the same as the two-centrifuge process described above for Examples 10-23, except that no water was added prior to mixer 60. The copper concentration of the light phase entering the second centrifuge was 2.2 ppm. The process variables were scaled process flow rate and light phase back pressure at the second centrifuge. The results, presented in Table 4, show that operating a second centrifuge in clarifier mode (that is, without aqueous phase addition between the first and second centrifuges, and with the second more dense phase discharged from the centrifuge) is effective to reduce the copper concentration of the poly(arylene ether) phase, that the efficiency of copper removal is generally increased at lower process rates, and that the efficiency of copper removal is generally increased at increased light phase back pressure at the second centrifuge.

TABLE 4

| | Ex. 24 | Ex. 25 | Ex. 26 | Ex. 27 |
|---|---|---|---|---|
| Light phase back pressure | 250 | 250 | 200 | 200 |
| (kPa) | | | | |
| Scaled process flow rate | 1.33 | 2.0 | 2.0 | 2.67 |
| Cu in light phase (ppm) | 0.4 | 0.6 | 0.8 | 0.4 |

### EXAMPLES 28-33

These examples illustrate the use of a coalescer as the second phase separator in a process with two phase separators. The process is the same as the two-centrifuge process depicted in FIG. 4 and described above for Examples 10-23, except that a prefilter (obtained from Pall Corporation) and coalescer (obtained as PhaseSep® from Pall Corporation) were substituted for the second centrifuge. This apparatus was tested at three different flow rates. Copper removal was expressed as a percent of the copper present in the light phase entering the prefilter. The results, presented in Table 5, show the coalescer is effective to remove some copper from the incoming process stream.

TABLE 5

| | Ex. 28 | Ex. 29 | Ex. 30 | Ex. 31 |
|---|---|---|---|---|
| Scaled process flow rate | 1.0 | 1.0 | 1.0 | 1.0 |
| Cu removal (%) | 41 | 55 | 66 | 78 |

| | Ex. 32 | Ex. 33 |
|---|---|---|
| Scaled process flow rate | 1.33 | 2.67 |
| Cu removal (%) | 56 | 79 |

### EXAMPLES 34-40

These experiments illustrate the use of decantation as an alternative method to effect the first separation. The initial process steps, up to and including combination of the aqueous chelant solution with the poly(arylene ether) reaction mixture, were the same as those described above in Examples 1-5, except that they were conducted on a laboratory scale. Before being combined with the aqueous chelant solution, the poly(arylene ether) reaction mixture had a copper concentration of 130 ppm. The combined aqueous chelant solution and poly(arylene ether) reaction mixture were allowed to stand at 70° C. for various “settling” times ranging from zero to three hours, before the light phase was decanted from the heavy (aqueous) phase, and the copper content of the decanted light phase was measured. The results, presented in Table 6, show that while decantation is an effective method to effect phase separation and thereby reduce the copper content of the poly(arylene ether) reaction mixture, the copper removal efficiency is dependent on the settling time, with settling times on the order of hours required to maximize copper removal.

TABLE 6

| | Ex. 34 | Ex. 35 | Ex. 36 | Ex. 37 |
|---|---|---|---|---|
| Settling time (hours) | 0 | 0.5 | 1 | 1.5 |
| Cu concentration (ppm) | 37 | 24 | 12 | 24 |

| | Ex. 38 | Ex. 39 | Ex. 40 |
|---|---|---|---|
| Settling time (hours) | 2 | 2.5 | 3 |
| Cu concentration (ppm) | 19 | 9 | 7 |

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

All ranges disclosed herein are inclusive of the endpoints, and the endpoints are independently combinable with each other.

The use of the terms “a” and “an” and “the” and similar referents in the context of describing the invention (especially in the context of the following claims) are to be construed to cover both the singular and the plural, unless otherwise indicated herein or clearly contradicted by context. Further, it should further be noted that the terms “first,” “second,” and the like herein do not denote any order, quantity, or importance, but rather are used to distinguish one element from another. The modifier “about” used in connection with a quantity is inclusive of the stated value and has the meaning dictated by the context (e.g., it includes the degree of error associated with measurement of the particular quantity).

1. A method of purifying a poly(arylene ether), comprising:
combining a chelant with a poly(arylene ether) reaction mixture comprising a poly(arylene ether), an aromatic hydrocarbon solvent, water, and a catalyst metal;
separating the combined chelant and poly(arylene ether) reaction mixture in a separation apparatus with an average residence time of less than or equal to 60 seconds to yield a first less dense phase comprising poly(arylene ether) and solvent and a first more dense phase comprising water and chelant;
combining the first less dense phase with an aqueous solution; and
separating the combined first less dense phase and aqueous solution in a separation apparatus with an average residence time of less than or equal to 60 seconds to yield a second less dense phase comprising poly(arylene ether) and solvent and a second more dense phase comprising water.

2. The method of claim 1, wherein the poly(arylene ether) reaction mixture comprises about 10 to about 40 weight percent of the poly(arylene ether), based on the total weight of the poly(arylene ether) reaction mixture.

3. The method of claim 1, wherein said combining a chelant comprises combining a chelant solution comprising about 5 to about 50 weight percent chelant, based on the total weight of the chelant solution.

4. The method of claim 3, wherein the chelant solution is added in an amount of about 0.05 to about 5 weight percent, based on the weight of the poly(arylene ether) reaction mixture.

5. The method of claim 3, wherein said combining a chelant solution comprises combining the poly(arylene ether) reaction mixture and the chelant solution in a stirred tank having a stirrer Reynolds number of about 10,000 to about 60,000.

6. The method of claim 3, wherein said combining a chelant solution comprises combining the poly(arylene ether) reaction mixture and the chelant solution with a mixing energy of about 1 to about 10 kilojoules per kilogram total of the poly(arylene ether) reaction mixture and the chelant solution.

7. The method of claim 3, further comprising recycling at least 30 weight percent of the second more dense phase for use as at least a portion of the chelant solution.

8. The method of claim 1, wherein the poly(arylene ether) reaction mixture is prepared by oxidative polymerization in a reactor, and wherein said adding a chelant comprises adding the chelant to the poly(arylene ether) reaction mixture in the reactor.

9. The method of claim 1, further comprising adding water to the poly(arylene ether) reaction mixture.

10. The method of claim 1, wherein said separating the combined chelant and poly(arylene ether) reaction mixture is conducted in about 4 to about 60 seconds.

11. The method of claim 1, wherein the aqueous solution is used in an amount of about 1 to about 8 weight percent, based on the weight of the first less dense phase.

12. The method of claim 1, wherein the aqueous solution is substantially free of chelant.

13. The method of claim 1, wherein the aqueous solution consists of water.

14. The method of claim 1, wherein the aqueous solution has a temperature of about 50 to about 70° C. when it is combined with the first less dense phase.

15. The method of claim 1, wherein the temperatures of the first less dense phase and the aqueous solution differ by less than 20° C. before they are combined.

16. The method of claim 1, wherein said combining the first less dense phase with an aqueous solution is conducted in a mixing device with an average residence time of about 2 to about 60 seconds.

17. The method of claim 1, wherein said combining the first less dense phase with an aqueous solution comprises combining a first less dense phase flow having a Reynolds number of about 1,000 to about 4,000 with an aqueous solution flow having a Reynolds number of about 100 to about 400; and wherein a ratio of the Reynolds number of the first less dense phase flow to the Reynolds number of the aqueous solution flow is about 5 to about 20.

18. The method of claim 1, wherein said combining the first less dense phase with an aqueous solution is conducted in about 2 to about 10 seconds.

19. The method of claim 1, wherein said combining the first less dense phase with an aqueous solution comprises using a static mixer.

20. The method of claim 1, wherein said separating the first less dense phase and aqueous solution is conducted in about 4 to about 60 seconds

21. The method of claim 1, wherein at least one of said separating the combined chelant and poly(arylene ether) reaction mixture and said separating the combined first less dense phase and aqueous solution comprises using a liquid-liquid centrifuge.

22. The method of claim 1, wherein at least one of said separating the combined chelant and poly(arylene ether) reaction mixture and said separating the combined first less dense phase and aqueous solution comprises operating a liquid-liquid centrifuge at a light phase back pressure of about 50 to about 300 kilopascals.

23. The method of claim 1, wherein the second less dense phase comprises the catalyst metal in a concentration of about 0.1 to about 2 parts per million by weight, based on the total weight of the second less dense phase.

24. The method of claim 1, wherein a ratio of catalyst metal concentration in the second less dense phase to catalyst metal concentration in the poly(arylene ether) reaction mixture is about 1:500 to about 1:50.

25. The method of claim 1, further comprising isolating the poly(arylene ether) from solution, wherein the poly(arylene ether) comprises less than 10 weight percent of particles smaller than 38 micrometers and less than or equal to 2 parts per million by weight of the catalyst metal.

26. A poly(arylene ether) prepared according to the method of claim 25.

27. The method of claim 1, wherein the poly(arylene ether) comprises repeating structural units having the formula 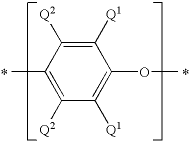
wherein for each structural unit, each Q¹ is independently halogen, unsubstituted or substituted C₁-C₁₂ hydrocarbyl with the proviso that the hydrocarbyl group is not tertiary hydrocarbyl, C₁-C₁₂ hydrocarbylthio, C₁-C₁₂ hydrocarbyloxy, or C₂-C₁₂ halohydrocarbyloxy wherein at least two carbon atoms separate the halogen and oxygen atoms; and each Q² is independently hydrogen, halogen, unsubstituted or substituted C₁-C₁₂ hydrocarbyl with the proviso that the hydrocarbyl group is not tertiary hydrocarbyl, C₁-C₁₂ hydrocarbylthio, C₁-C₁₂ hydrocarbyloxy, or C₂-C₁₂ halohydrocarbyloxy wherein at least two carbon atoms separate the halogen and oxygen atoms.

28. The method of claim 1, wherein the poly(arylene ether) comprises 2,6-dimethyl-1,4-phenylene ether units, 2,3,6-trimethyl-1,4-phenylene ether units, or a combination thereof.

29. The method of claim 1, wherein the poly(arylene ether) comprises a bifunctional poly(arylene ether) having the structure 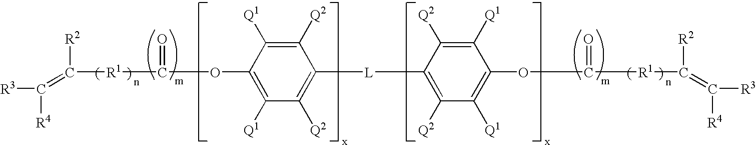
wherein each occurrence of Q¹ is independently halogen, unsubstituted or substituted C₁-C₁₂ hydrocarbyl with the proviso that the hydrocarbyl group is not tertiary hydrocarbyl, C₁-C₁₂ hydrocarbylthio, C₁-C₁₂ hydrocarbyloxy, or C₂-C₁₂ halohydrocarbyloxy wherein at least two carbon atoms separate the halogen and oxygen atoms; each occurrence of Q² is independently hydrogen, halogen, unsubstituted or substituted C₁-C₁₂ hydrocarbyl with the proviso that the hydrocarbyl group is not tertiary hydrocarbyl, C₁-C₁₂ hydrocarbylthio, C₁-C₁₂ hydrocarbyloxy, or C₂-C₁₂ halohydrocarbyloxy wherein at least two carbon atoms separate the halogen and oxygen atoms; each occurrence of x is independently 0 to about 100, with the proviso that the sum of each occurrence of x is at least three; each occurrence of R¹ is C₁-C₁₂ hydrocarbylene; each occurrence of m is independently 0 or 1; each occurrence of n is independently 0 or 1; each occurrence of R²-R⁴ is independently hydrogen or C₁-C₁₈ hydrocarbyl; and L has the structure 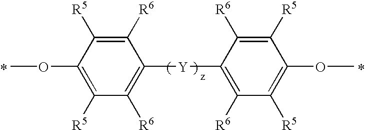
wherein each occurrence of R⁵ and R⁶ is independently hydrogen, halogen, unsubstituted or substituted C₁-C₁₂ hydrocarbyl with the proviso that the hydrocarbyl group is not tertiary hydrocarbyl, C₁-C₁₂ hydrocarbylthio, C₁-C₁₂ hydrocarbyloxy, or C₂-C₁₂ halohydrocarbyloxy wherein at least two carbon atoms separate the halogen and oxygen atoms; z is 0 or 1; and Y has the structure 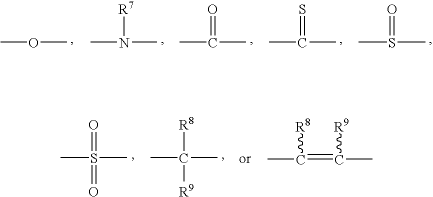
wherein R⁷, R⁸, and R⁹ are each independently hydrogen, C₁-C₁₂ hydrocarbyl, or the like.

30. The method of claim 1, wherein the aromatic hydrocarbon solvent is selected from the group consisting of benzene, toluene, xylenes, chlorobenzene, dichlorobenzenes, trichlorobenzenes, tetrachlorobenzenes, pentachlorobenzene, hexachlorobenzene, and combinations thereof.

31. The method of claim 1, wherein the aromatic hydrocarbon solvent is toluene.

32. The method of claim 1, wherein the catalyst metal is selected from the group consisting of copper, manganese, cobalt, and mixtures thereof.

33. The method of claim 1, wherein the catalyst metal is copper.

34. The method of claim 1, wherein the chelant is selected from the group consisting of polyalkylenepolyamine polycarboxylic acids, aminopolycarboxylic acids, aminocarboxylic acids, polycarboxylic acids, alkali metal salts of the foregoing acids, alkaline earth metal salts of the foregoing acids, mixed alkali metal-alkaline earth metal salts of the foregoing acids, and combinations thereof.

35. The method of claim 1, wherein the chelant is an alkali metal salt of nitrilotriacetic acid.

36. A method of purifying a poly(arylene ether), comprising:
combining a chelant with a poly(arylene ether) reaction mixture comprising toluene, copper catalyst metal, and a poly(arylene ether) comprising 2,6-dimethyl-1,4-phenylene ether units; wherein the chelant is provided as a chelant solution comprising an alkali metal salt of nitrilotriacetic acid;
separating the combined chelant and poly(arylene ether) reaction mixture in a liquid-liquid centrifuge with an average residence time of about 4 to about 40 seconds to yield a first less dense phase comprising poly(arylene ether) and solvent and a first more dense phase comprising water and chelant;
combining the first less dense phase with an aqueous solution consisting of water in about 2 to about 60 seconds; and
separating the combined first less dense phase and aqueous solution in about 4 to about 40 seconds with a liquid-liquid centrifuge to yield a second less dense phase comprising poly(arylene ether) and solvent and a second more dense phase comprising water.

37. The method of claim 36, further comprising isolating the poly(arylene ether) from solution, wherein the poly(arylene ether) comprises less than 10 weight percent of particles smaller than 38 micrometers and less than or equal to 2 parts per million by weight of copper.

38. A poly(arylene ether) prepared according to the method of claim 37.

39. A method of purifying a poly(arylene ether), comprising:
combining a chelant with a poly(arylene ether) reaction mixture comprising toluene, copper catalyst metal, and a poly(arylene ether) comprising 2,6-dimethyl-1,4-phenylene ether units; wherein the chelant is provided as a chelant solution comprising an alkali metal salt of nitrilotriacetic acid;
separating the combined chelant and poly(arylene ether) reaction mixture in a liquid-liquid centrifuge with an average residence time of about 4 to about 15 seconds to yield a first less dense phase comprising poly(arylene ether) and solvent and a first more dense phase comprising water and chelant; wherein the liquid-liquid centrifuge is operated at a light phase back pressure of about 50 to about 300 kilopascals;
combining the first less dense phase with an aqueous solution consisting of water in about 2 to about 60 seconds; wherein the aqueous solution is used in an amount of about 1 to about 8 weight percent, based on the weight of the first less dense phase; and
separating the combined first less dense phase and aqueous solution in a liquid-liquid centrifuge with an average residence time of about 4 to about 15 seconds to yield a second less dense phase comprising poly(arylene ether) and solvent and a second more dense phase comprising water; wherein the liquid-liquid centrifuge is operated at a light phase back pressure of about 50 to about 300 kilopascals.

40. The method of claim 39, further comprising isolating the poly(arylene ether) from solution, wherein the poly(arylene ether) comprises less than 10 weight percent of particles smaller than 38 micrometers and less than or equal to 2 parts per million by weight of copper.

41. A poly(arylene ether) prepared according to the method of claim 40.
